(19) United States
(12) Patent Application Publication
(10) Pub. No.: US 2016/0051413 A1
(43) Pub. Date: Feb. 25, 2016

(54) WOUND CARE FOOT WRAP

(71) Applicant: Ansell Limited, Richmond (AU)

(72) Inventors: Jeffrey C. Moreland, Simpsonville, SC (US); Roger E. Huckfeldt, Nixa, MO (US); Eric Thompson, Central, SC (US); Jamie Ashworth, Freehold, NJ (US); Kristin King, West New York, NJ (US); Carrie Lirae Merck, Clemson, SC (US)

(21) Appl. No.: 14/835,207

(22) Filed: Aug. 25, 2015

Related U.S. Application Data
(60) Provisional application No. 62/041,171, filed on Aug. 25, 2014.

(51) Int. Cl. A61F 13/06; A61F 13/00

(52) U.S. Cl. CPC A61F 13/064; A61F 13/00021

(57) ABSTRACT

Anti-microbial wound care foot wraps, which are wrapped around the foot and/or lower leg of a patient, and outer dressings are disclosed.

### CROSS-REFERENCE TO RELATED APPLICATIONS

This application claims the benefit of U.S. Provisional Application No. 62/041,171, filed on Aug. 25, 2014, which is incorporated herein in its entirety.

### BACKGROUND

1. Field of the Invention

Embodiments of the present invention generally relate to wound care articles and, more particularly, to wound care foot wraps, optionally comprising moisture-wicking yarns and/or antimicrobial agents and/or outer dressings. Methods of manufacturing the wound care foot wraps are also disclosed.

2. Description of the Related Art

Wounds, such as cuts, abrasions, and burns and chronic wounds, such as lesions and ulcers caused by dermatitis, contact dermatitis, vasculitis, psoriasis, diabetes, as well as patients suffering from pressure ulcers, i.e., as a consequence of paralysis, require constant care and special medical treatments. Treatment of these many types of wounds poses unique problems for medical personnel. On one hand, moisture and air are required to allow wounds to heal for most but not all wounds. However, the healing of some wounds, particularly chronic wounds, can be slowed by the presence of too much moisture, such as a wound exudate. Conversely, wounds of this type are acutely susceptible to infection and therefore must be insulated from germs, bacteria, viruses, and other pathogens.

Gauzes and other medical dressings, which are typically woven structures, have been used to treat cuts, abrasions, and chemical-, heat- and flame-caused burns. However, treatment often entails multiple changes of dressings, which takes a lot of time and is further disfavored because changing dressings often means abrading the surface of the wound, delaying healing. Further still, some wounds require compression-type therapies, which may require an additional device. In addition, particularly for wounds on or near joints, such as ankles and knees, many wearable articles consist of rigid, stiff fabrics leading to stress and irritation during donning, usage, and doffing.

Therefore, there is a need in the art for anti-microbial wound care apparatus for feet, ankles, and lower leg extremities, such as a foot and/or leg wrap, that manages moisture, exudate, and perspiration, are flexible for a tight and comfortable fit during mobility, are donned and doffed easily, and promote healing by allowing intimate contact of the foot wrap having the antimicrobial with the skin of the wearer without sticking to or abrading the skin/wound.

### SUMMARY

Embodiments of the invention include wound care articles substantially as shown in and/or described in connection with at least one of the figures, as set forth more completely in the claims. Various advantages, aspects, and novel features of the present disclosure, as well as details of an exemplary embodiment thereof, will be more fully understood from the following description and drawings.

### BRIEF DESCRIPTION OF THE DRAWINGS

So that the manner in which the above recited features of the present invention can be understood in detail, a more particular description of the invention, summarized above, may be had by reference to embodiments, some of which are illustrated in the appended drawings. It is to be noted that the appended drawings illustrate typical embodiments of this invention and are not to be considered limiting of its scope, for the invention admits to other equally effective embodiments. It is to be understood that elements and features of one embodiment may be in other embodiments without further recitation. Also, where possible, identical reference numerals have been used to indicate comparable elements common to the figures.

FIG. 1 depicts a top view of a flattened unsewn foot wrap according to embodiments of the invention;

FIG. 2 depicts a top view of a sewn foot wrap, according to embodiments of the invention;

FIG. 3 depicts a perspective view of the foot wrap of FIG. 2 wrapped around a foot, according to embodiments of the invention;

FIG. 4 depicts a perspective view of a toe separator, according to embodiments of the invention;

FIG. 5 depicts a bottom view of a bare foot having the toe separator of FIG. 4 thereon, according to embodiments of the invention;

FIG. 6 depicts a perspective view of the foot and toe separator thereon of FIG. 5, according to embodiments of the invention; and

FIG. 7 depicts a plan view of a yarn used in embodiments according to the invention.

### DETAILED DESCRIPTION

Embodiments of the invention include a foot wrap for wound care and methods for making wearable, easy to don and doff anti-microbial foot wraps substantially as shown in and/or described in connection with at least one of the figures, as set forth more completely in the claims. The foot wrap for the treatment of wounds comprises a knitted fabric layer having anti-microbial properties and a moisture wicking fiber and/or an elastic yarn. Embodiments of the invention comprise foot wraps having flaps capable of adjusting compression of a body part placed therein, i.e., foot wraps may be loose fitting and releasably tightened to view wounds, and the progress of healing, easily and without contacting the wound itself, and re-wrapping the foot and/or leg without changing the foot wrap. Embodiments of the invention comprise multiple flaps, which can be tightened to produce graduated amounts of pressure, e.g., tightest around a foot, looser around an ankle, and loosest above the ankle or calf, providing a compression therapy that can be adjusted as appropriate.

Any embodiment according to the invention disclosed herein can promote the healing of wounds, such as chronic wounds, e.g., venous ulcers, vascular and arterial ulcers, Stage I-IV dermal ulcers, diabetic ulcers, lesions and ulcers caused by dermatitis, contact dermatitis, vasculitis, psoriasis, etc., as well as patients suffering from pressure ulcers, burns, cuts, and/or abrasions.

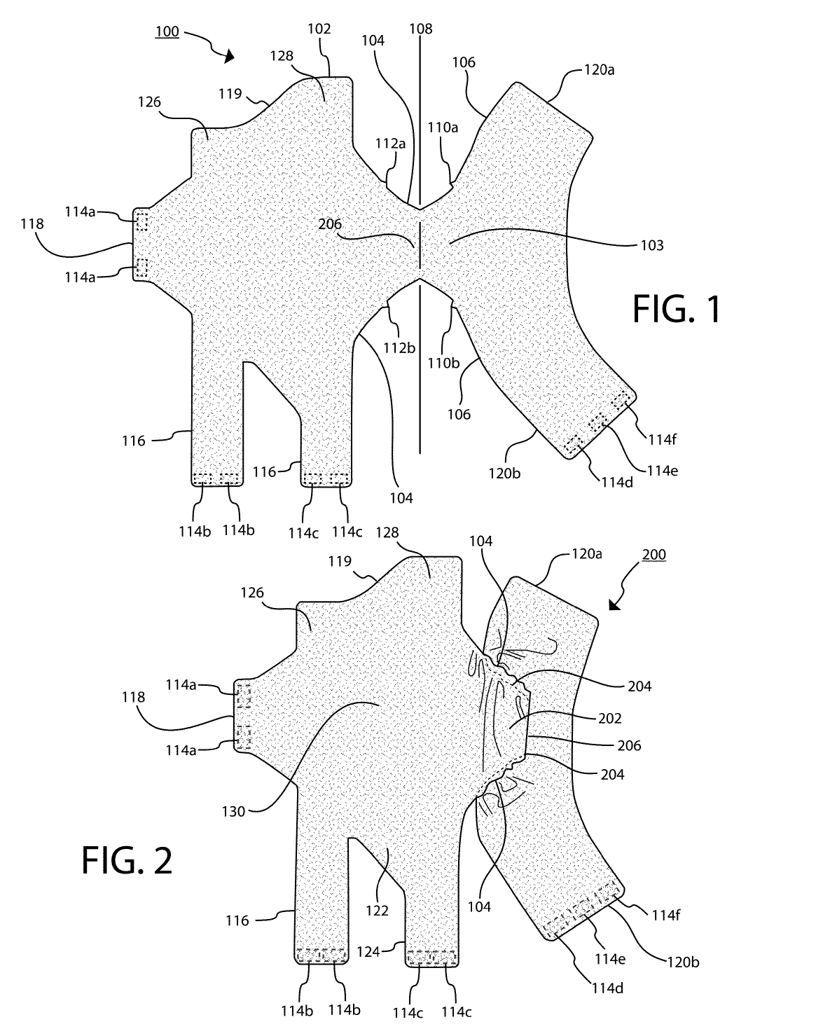

FIG. 1 depicts a top view 100 of a flattened unsewn foot wrap 102 according to embodiments of the invention. The wound care foot wrap 102 is either a woven or knitted article and comprises a fabric having an anti-microbial yarn. In some embodiments according to the invention, the wound care foot wrap 102 comprises a metalized yarn. For example, a nylon yarn having a metal infused therein, such as silver, gold, zinc, copper, and their alloys. Other examples according to embodiments of the invention comprise a silver-coated nylon 6,6 yarn, such as yarns manufactured by Noble Biomaterials of Scranton, PA, such as an X-static® brand yarn. Alternatively, the fabric comprises a yarn containing an anti-microbial manufactured by Iftna, Inc. of Ontario, Canada. The wound care foot wrap 102 comprises fold points at a heel portion 103 (generally correlating with an area that a heel would be if the foot wrap were on a foot). The wound care foot wrap 102 is folded along a plane 108 running through the wound care foot wrap 102.

The wound care foot wrap 102 further comprise first tabs 110a and 110b which mate with corresponding second tabs 112a and 112b, when the wound care foot wrap 102 is folded along plane 108 and are sewn together, which are for a seamstress or clinician to use as a guide so that the wound care foot wrap 102 is sewn reproducibly, creating a heel pocket, discussed below. For example, the first tab 110a and the corresponding second tab 112a are sewn together and the first tab 110b and the corresponding second tab 112b are sewn together. The wound care foot wrap 102 further comprises inner sew borders 106 and outer sew borders 104. Embodiments of the invention include where the inner sew borders 106 and outer sew borders 104 are mirror images of each other or, alternatively, where the outer sew borders 104 comprise a parabolic, concave shape (as shown). As discussed below, embodiments of the invention where the inner sew borders 106 and outer sew borders 104 are essentially mirror images of each other comprise a near symmetrical seam while where the inner sew borders 106 comprise a different shape, e.g., a parabolic, concave shape, an asymmetrical seam is created. The wound care foot wrap 102 comprises one or more pieces of fabric and in some embodiment is a single piece construction of fabric. Furthermore, it can be easily customized for specific medical conditions. In other words, the fabric can be cut out easily where it may need to be, i.e., where it is desirable to do so, for example, because an area needs to be ventilated, no wound was ever present, or a wound has healed.

Embodiments according to the invention also comprise wherein the wound care foot wrap 102 comprises fasteners 114, such as hooks, for example, the hooks side of a VELCRO® fastener. As shown, the fasteners 114 are disposed on a plurality of flaps, e.g., a foot flap 116, a toe flap 118, an instep flap 124, and lower leg flaps 120a and 120b. As shown, the foot flap 116, the toe flap 118, and the instep flap 124 each have two fasteners 114 (114b, 114a, and 114c respectively). However, embodiments according to the invention comprise wherein the foot flap 116, the toe flap 118, and the instep flap 124 each have only one fastener 114 or more than two fasteners 114. Similarly, the lower leg flap 120a and 120b comprise one, two, three or more fasteners 114. The foot flap 116, the toe flap 118, the instep flap 124, and the lower leg flaps 120a and 120b may be shorter (i.e., so that they traverse, for example, one quarter to one half way around a foot wrapped therewith) or longer (i.e., so that they traverse, for example, one half to completely around a foot, ankle, or lower leg wrapped therewith). For example, if the foot flap 116, the instep flap 124, and/or the lower leg flaps 120a, and 120b are longer, they can be significantly tightened, creating a compression foot wrap. If the foot flap 116, for example, is shorter, it may be capable of engaging the wound care foot wrap 102 less tightly, i.e., because it does not traverse an area on the opposite surface of a foot and cannot be held as tightly. In either case, the loops side of a hook and loops fastener is optional. In other words, the hooks of the fastener(s) 114 can engage the loops of a knitted article or the interstices of a woven article, thereby obviating the need for the loops part of a hooks-and-loops fastener. This is a significant advance because the hooks on the foot flap 116, the toe flap 118, the instep flap 124, and the lower leg flaps 120a and 120b can engage any areas of the foot wrap 102, allowing medical personnel to engage various areas while loosening other areas as needed. Moreover, having multiple flaps allows many variable tightening/loosening configurations for graduated compressions, as discussed below. Also, embodiments according to the invention comprise wherein the areas around the plane 108 are cut out to create a more three-dimensional area for the heel of a patient, e.g., a heel pocket.

FIG. 2 depicts a top view 200 of a sewn foot wrap 102, according to embodiments of the invention. The wound care foot wrap 102 is shown sewn with stitches 204, creating a heel pocket 202, after the first tabs 110a and 110b and the second tabs 112a and 112b are matched and sewn with stitches 204. As shown, the stitches 204 traverse to an edge 206, allowing a loose fitting that allows the sewn foot wrap 102 to be easily put on and taken off the foot of a patient. For example, the sewn foot wrap 102 comprises a main area 130, which has a surface area roughly the size of a large foot, including surface area for the bottom of a foot, around the sides of a foot, and the top and instep of a foot, i.e., approximately 12-16 inches in length and 10-12 inches in width. Extending from the upper area 126 of the main area 130 is the toe flap 118, which is approximately 4-6 inches long. Extending from a lower area 122 of the main area 130 is one or more flaps, such as the foot flap 118 and/or the instep flap 124. Extending from an upper area 126 of the main area 130 is a top flap 119, which is capable of being folded over a top of a foot, which can in turn be engaged by the foot flap 116, the instep flap 124, and/or the toe flap 118. Extending from a position between the lower area 122 and the upper area 128 of the main area 130 is the heel portion 103 and the heel pocket 202, which comprises a circumference of approximately four to six inches. Extending rearward of the heel pocket 202, and in some embodiments extending arcuately, are the lower leg flaps 120a and 120b, the lower leg flap 120a adjacent to the instep flap 124 and/or lower area 122 and the lower leg flap 120b adjacent to the upper area 128.

The heel pocket 204 has stitches 204 that join the main area 120 with the heel portion 103. Also, the shorter that the stitches 204 are, the larger the heel pocket 202 becomes, allowing easy customization for different sized feet. The foot flap 116, the toe flap 118, the instep flap 124, and the lower leg flaps 120a and 120b, having the fasteners 114, are shown outstretched, i.e., not engaged with any part of the sewn foot wrap 102. However, when the foot flap 116, the toe flap 118, the instep flap 124, and the lower leg flaps 120a and 120b are engaged with another part of the sewn foot wrap 102, such as areas 126, 130, 128, the foot flap 116, the toe flap 118, the instep flap 124, and the lower leg flaps 120a and 120b can be engaged as tightly or as loosely, without having the loops portion of the fastener(s) 114 in a specific area, as needed for any particular medical situation. For example, the foot flap 116, the toe flap 118, the instep flap 124, and the lower leg flaps 120a and 120b are optionally long enough to traverse around the foot or lower leg of a patient and engage the wound care foot wrap 102 in any area, so many sizes, fits, and tensions/compressions for different patients or, also, for the same patient as swelling recedes, may be accommodated. Moreover, because the wound care foot wrap 102 is wrapped around a foot and/or lower leg of a patient, it can be as tight or as loose as needed and simultaneously maintain flexible contact with a wound.

In practice, the heel of a foot would be placed on or within the heel pocket 202. The first area 119 would be stretched or disposed across an instep of a foot of a patient. The foot flap 116, the instep flap 124 and the lower area 122 would next be stretched across the first area 119 and secured thereto with the hooks of the fasteners 114 of any flap. The toe flap 118 would then be stretched across from toes of the foot toward the instep and secured to the main area 130 with the hooks of the fasteners 114a, which are disposed on the foot flap 118. Thereafter, the lower leg flap 120b would be stretched across, partially around, or completely around the ankle of a patient. The lower leg flap 120b would then be wrapped around the ankle and secured thereto with the hooks of the fasteners 114d, 114e, and/or 144f disposed on the lower leg flap 120b.

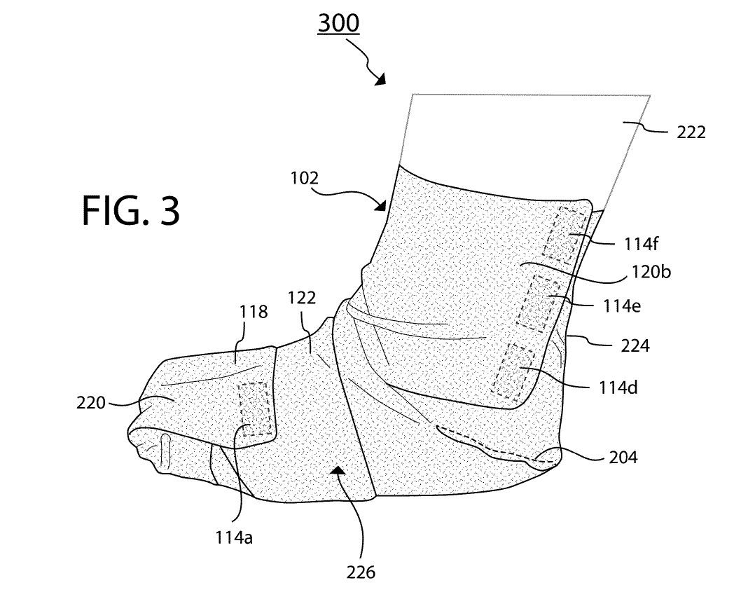

FIG. 3 depicts a perspective view 300 of the foot wrap of FIG. 2 wrapped around a foot, according to embodiments of the invention. The perspective view 300 shows the wound care foot wrap 102 from left side foot perspective. The wound care foot wrap 102 is depicted as wrapped around a foot and leg 222 and tightened thereto with the fasteners 114a in a toe area 220, an instep area 226, and an ankle area and/or lower leg area 224. As discussed above, because the wound care foot wrap 102 is loose fitting and releasably tightened with the hooks of the fasteners 114a, 114b, 114c, 114d, 114e, and/or 114f, the wound care foot wrap 102 may be removed to view wounds, and the progress of healing, easily and without contacting the wound itself, and re-applied. For example, a gradient of pressure may be created, such as for a diabetic wound, wherein the fastener 114a, which adheres the toe flap 118, and the foot flap 116, which is adhered by the fasteners 114b are wrapped and held tightly. The instep flap 124, having fasteners 114c, may be wrapped and held somewhat less tightly, while the lower leg flap 120b, having fasteners 114d, 114e, and 114f are wrapped and held looser from 114d to 114f, creating larger amounts of pressure near the toes of a patient and having gradually less pressure towards the knee of a patient. Also, in some embodiments according to the invention, there may be cuts between the fasteners 114d and 114e as well as between 114e and 114f, so that fasteners 114d, 114e, and 114f can be tightened or loosened with even greater ease.

Some embodiments further comprise variable plaiting so that more elastomeric yarn is disposed near the toes, for example, plaiting an elastomeric yarn along with a main yarn every course or every other course near a portion of the foot wrap where toes would be wrapped, and lesser amounts, e.g., a plaited elastomeric yarn approximately every fourth course in portions that wrap the ankle, and even lesser amounts, e.g., every sixth or eight course, in portions that wrap the calf or lower leg, which allows clinicians to exploit a more adjustable compression of the foot wrap while remaining tight and comfortable. Also, as wounds heal, and less pressure is needed, the foot wrap need not be replaced because it remains capable of producing a wide range of compression as appropriate for any given therapy.

Embodiments of the invention can manage moisture, i.e., keep a wound moist but not wet, which can both inhibit bacterial growth and/or manage toxins emanating from the wound itself. Embodiments of the invention can wick moisture in all three axes, i.e., around the circumference of a leg, along a longitudinal axis of a leg, i.e., from the ankle towards the knee, and/or from a surface of skin to, for example, a first layer (skin-contacting) of the wrap and, optionally, to a second layer, e.g., an outer dressing. Embodiments according to the invention comprise additional knitted layers.

Knitted or woven foot wraps may comprise more than one yarn. In some embodiments of the invention, a first layer is an absorbent, moisture-wicking knitted layer having an antimicrobial layer for the treatment of wounds and a second layer comprises cotton, polyesters, nylons, or an elastic yarns, such as SPANDEX® or LYCRA®, and the like and/or any combination of these yarns. Cotton may be used, for example, to absorb moisture or other fluids, such as exudate or blood. Elastic yarns impart stretch and comfort properties, allowing variable tightening and loosening as needed. Moreover, foot wraps can be specified to use specified amounts of elastic yarns that can enhance stretching of the foot wrap, so that one size may fit all users and/or can allow easy tightening and loosening as needed for specific wounds.

In some embodiments, the foot wrap comprises one layer, i.e., a first layer, which contacts the skin of the wearer, and comprises metals, such as silver, gold, copper, or zinc, or their alloys or combinations of elemental metals and alloys or other substances having antimicrobial properties, which promotes healing of the wound of a wearer and promotes an antiseptic environment. In some embodiments, the foot wraps comprise a second, third, or fourth layer disposed on the first layer, each comprising cotton, polyesters, nylons, SPANDEX®, hydrophilic, and anti-microbial yarns (such as metalized yarns or a yarn having anti-microbial treatments disposed thereon). In some embodiments of the invention, a nylon yarn, for example, is used as a main yarn, while in other embodiments, nylon yarn may be plaited into a non-nylon main yarn. In other embodiments of the invention, two or more yarns are plaited throughout the article and, accordingly, the article has two layers throughout the entire article. The skin-contacting layer and outer layers may optionally comprise the same nylon yarn, a different nylon, or a non-nylon yarn. Some yarns are treated with TRIOSYN®, triclosan, 2-propanol, quaternary ammonium compounds, n-halamines, or compounds and combinations thereof, for their antimicrobial properties. Silver-zinc and silver-copper zeolites are also suitable antimicrobials, as well as other anti-microbials known to those in the art, such as, but not limited to, polymeric biguanides, i.e., chlorhexidine gluconate.

In some embodiments, the foot wrap comprises a hydrophilic yarn such as a nylon yarn, such as nylon 6,6 yarn, having irregular cross-sections for exceptional wicking properties. Nylons promote the transport of moisture from the wound to the outer layer. One such example of a yarn having an irregular cross-section for wicking properties is Nilit® AQUARIUS, which, in addition to wicking moisture and exudate from a wound more effectively and efficiently, also comprises more volume between strands, which can contain more of an anti-microbial agent. Also, micro-denier and multi-filament yarns also promote superior wicking action. Hydrophilic yarns allow the transfer of metal ions from the yarn to the wound or eluent, promoting healing of the wound. Non-stick yarns are also contemplated according to embodiments of the invention. For example, any of the yarns disclosed herein may be blended with low surface tension yarns, such as modified polytetrafluoroethylene yarns and/or polyethylenes, to create yarns having enhanced non-stick properties. It is to be noted that any yarn discussed herein may comprise a hemostatic fiber therein. For example, a hemostatic fiber, i.e., a fiber causing blood to coagulate, such as treated cotton, may be employed on a covering yarn that contacts the wound.

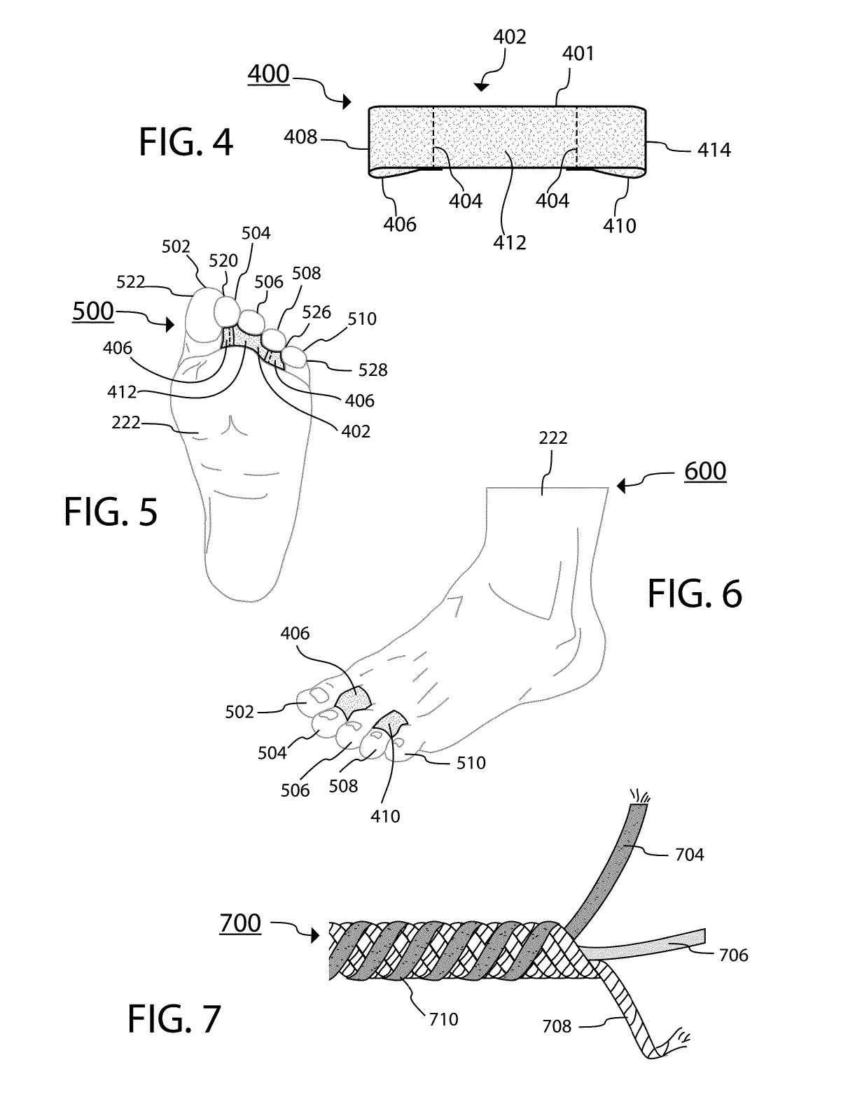

FIG. 4 depicts a perspective view 400 of a toe separator 402, according to embodiments of the invention. The toe separator 402 includes a strip 401 comprising a yarn and is folded back against itself at folds 408 and 414 and stitched with stitches 404, creating a first toe pocket 406 and a second toe pocket 410 and a median area 412 disposed therebetween. Embodiments according to the invention comprise wherein the toe separator 402 includes a metalized yarn, as discussed above with respected to the wound care foot wrap and further comprises a hydrophilic yarn such as a nylon yarn, such as nylon 6,6 yarn, having irregular cross-sections for exceptional wicking properties. Moreover, the toe separator 402 may be knitted with any covered yarn discussed herein.

FIG. 5 depicts a bottom view 500 of a foot and leg 222 having the toe separator 402 of FIG. 4 thereon, according to embodiments of the invention. As shown, the first toe pocket 406 of the toe separator 402 has a second toe 504 placed therein and the second toe pocket 410 has a ring toe 508 placed therein. The median area 412 is disposed under the third toe 506. In practice, the median area 412 could also be disposed on top on the third toe 506, for example, if there was a wound on top of the third toe 506 but no wound on its underside. Having the toe separator 402 disposed as above may offer the most efficient manner. For example, second toe 504 and ring toe 508 are both enveloped, and therefore in contact with an antimicrobial, within the toe separator 402. The fold 408 contacts an inside surface 520 of a big toe 502. Similarly, the fold 414 of the toe separator 402 contacts an inside surface of a pinky toe 510. And, when the toe separator 402 is used in conjunction with the wound care foot wrap 102, discussed above, external surfaces 522 and 528 of the big toe 502 and the pinky toe 510, respectively, would be in contact with the wound care foot wrap 102 and, therefore, all surfaces of all toes would be in contact with an antimicrobial on either the foot wrap 102 or the toe separator 402. In practice, however, the toe separator 402 could also be placed on any two toes.

FIG. 6 depicts a perspective view 600 of the foot 222 and the toe separator 402 thereon of FIG. 5, according to embodiments of the invention. As can be seen, the toe separator 402 has second toe 504 placed in toe pocket 406 and the ring toe 508 is placed in the toe pocket 410. As shown, the toe protector 402 has two toe pockets 406 and 410. However, a second strip of fabric (not shown) could be sewn onto the toe separator 402, creating a third toe pocket so that, for example, the second toe 504, the third toe 506, and the ring toe 508 were all completely enveloped within toe pockets.

FIG. 7 depicts a plan view of a yarn 700 used in embodiments according to the invention. The yarn 700 comprises a covered yarn. For example, an elastomeric yarn or filament 706, such as SPAN DEXO OR LYCRA®, is a core for the yarn 700. The elastomeric yarn or filament 706 has an absorbent fiber, yarn, or filament 708 wrapping the core, such as cotton. As shown, the absorbent fiber, yarn, or filament 708 is twisted. The absorbent fiber, yarn, or filament 708 is then covered with a nylon yarn for moisture management properties. Optionally, the nylon yarn, which most closely contacts the skin, further comprises an anti-microbial coating 710. The anti-microbial coating may be any of the anti-microbial coatings discussed herein. Embodiments of the covered yarn 700 include wherein the anti-microbial coating is elemental silver, which, in addition to providing anti-microbial properties, also promotes healing of wounds.

The following yarn and/or knitting structures permit the foot wraps made therewith to achieve absorbency (cotton), stretch (elastomeric yarn), and wound healing/anti-microbial (silver coating) and moisture management (highly wicking nylon yarns), any of which may be used to knit a foot wrap or an outer dressing, as discussed further below. At least one yarn comprises a core having a 354 denier (dn) (30/2 CC) cotton and 40 dn elastomeric yarn having a covering that comprises a 70 dn nylon 6,6, optionally further comprising silver coated thereon. At least one embodiment comprises elemental silver coated on a nylon yarn. At least one yarn comprises a core having 354 dn (30/2 CC) cotton and a 40 dn elastomeric yarn, and a first cover comprising 70 dn nylon 6,6 and a second cover yarn comprising a 70 dn silver coated nylon, such as an X-Static yarn. Also, at least one outer dressing and/or foot wrap and/or knitted article, such as a sock, comprises a yarn having a core comprising 354 dn (30/2 CC) cotton, and a 40 dn elastomeric yarn, and a cover yarn comprising 70 dn nylon 6,6, which is knitted with a plating yarn comprising a 70 dn silver coated nylon yarn, e.g., X-Static nylon.

Embodiments according to the invention further include outer dressings. The outer dressing is for use in conjunction with a wound care foot wrap 102 and/or the toe separator 402, as discussed above. Embodiments according to the invention also comprise wherein the outer dressing is a single seamless layer, substantially similar to the wound care foot wrap 102 itself. In other words, the outer dressing is optionally designed to have fasteners 114 located on flaps, such as the foot flap 116, the toe flap 118, and the lower leg flaps 120a and 120b of the wound care foot wrap 102 as well as a seamless construction located near the point of the ankle area that bends from side to side and from up and down. As with the wound care foot wrap 102, the flaps of the outer dressing are optionally long enough to traverse at least half way around the foot of a patient, so the flaps can accommodate many sizes, fits, and tensions/compressions for different patients or, also, for the same patient as swelling recedes. Embodiments of the invention further include a two-piece construction, having two halves, which are adhered to each other releasably with fasteners, such as hook-and-loop fasteners, such as VELCRO®. The hook fasteners on the outer dressing may engage a loops aspect of a fastener located on the wound care foot wrap 102 or, alternatively, may engage and be secured with the loops or interstices of the wound care foot wrap 102 itself.

The outer dressing may be a knitted article or a woven fabric, substantially similar to articles disclosed herein, including the incorporation of silver, silver alloys, or other metals in yarns for anti-microbial effects. Specifically, an outer dressing having a yarn incorporating silver, as discussed herein, provides a barrier from outside pathogens from penetrating to the article disposed nearer the skin. And, as the article disposed on the skin comprises a yarn incorporating silver thereon protects against pathogens already on the skin or emanating therefrom, a dual barrier, i.e., internal and external, is created. In some embodiments, the outer dressing is a 10-18 gauge knit. In some embodiments, it is a 13 gauge knit. Where a denser outer dressing is desirable, such as to absorb greater amounts of moisture, embodiments of the invention comprise an 18 gauge knit. In some embodiments, the yarn used to manufacture any skin-contacting article discussed herein is a silver-coated nylon 6,6 yarn, which is, for example, between 140 and 221 denier, which is capable of being knit with an 18 gauge needles, such as an X-static® yarn manufactured by Noble Biomaterials of Scranton, PA. In at least one embodiment according to the invention, a 13 gauge or 15 gauge needle(s) is used, for foot wraps for treating certain wounds requiring exposure to oxygen for healing. Without intending to be bound by theory, it is believed that a 13 gauge or 15 gauge knitted structure, which has larger interstices between the courses of yarn loops, which may be used for knitting yarns from 300 to 600 denier or larger, allows greater amounts of oxygen to reach wounds covered therewith. Alternatively, the skin-contacting article comprises a fabric containing an anti-microbial manufactured by Iftna, Inc. of Ontario, Canada. The outer dressing may also comprise a cotton fiber, rayon, a rayon/cotton blend, or the like, releasably placed over the wound care foot wrap. An outer dressing having cotton provides moisture absorption while the rayon is lubricious so that the outer dressing slides easily over the wound care foot wrap discussed above.

The outer dressing further comprises a highly elastic yarn, such as SPANDEX® or LYCRA®, so that, when the outer dressing is stretched during use, it clamps onto the primary article, i.e., a wound care foot wrap, which holds the wound care foot wrap in place and tightly to the skin of a patient. As above, regarding the wound care foot wrap, the outer dressing may comprise highly-wicking yarns, including, for example, STA-COOL® polyester, ringspun hydrophilic polyester HYDROTEC®, or DRYENERGY® polyester/cotton and may further comprise any shape for the treatment of a limb or body part, such as a leg, arm, knee, ankle, foot, waist, and the like. Micro-denier, multi-filament yarns, and yarns having non-circular, irregular cross-sections are particularly effective at wicking moisture away from one area, such as a wound, to other areas.

Embodiments of the invention may further comprise wherein the wound care foot wrap 102, the toe separator 402, or the outer dressing is knitted in accordance with the Knitted Variable Stitch Design (KVSD) and/or three-dimensional, Automatic-Knit-Liner technologies as is disclosed in commonly-assigned U.S. Pat. Nos. 6,962,064; 7,213,419; 7,246,509; 7,434,422; and 7,555,921, each of which is hereby incorporated by reference in its entirety. KVSD programming of knitted articles allows for areas of additional stretch, reinforcement, and building up of additional yarns in any area of the knitted structure, e.g., a foot wrap. For example, varying the stitch dimensions, such as yarn tension and needle depth, can produce articles having different levels of stretchability. The tension of the yarn may be varied by adjusting the tension of the yarn between a pinch roller and a knitting head by computer control of a knitting machine, as is disclosed in commonly-assigned U.S. Pat. No. 7,434,422. A layer that is knitted tighter will have less stretchability. Varying the depth of penetration of the knitting needle into the article, and by casting off or picking up additional stitches in a knitted course, can also affect stretchability. A shallower needle penetration produces an article that is tighter and more difficult to stretch.

Furthermore, foot wraps, toe separators, and outer dressings in accordance with embodiments of the invention can be knit with a knitting machine according to instructions provided via computer programming. Double-layered zones for knitted articles may be formed using a variable plaiting process, increasing the stretch in key flex areas of the gloves by altering the number of plaited courses in each section, such as the knuckles or the crotch between the index finger and thumb. For example, stretchable multi-layer functional zones are formed by plaiting a second yarn, such as an elastic yarn, such as SPANDEXO or LYCRA®, every fourth course in areas of low flex of the outer layer. Furthermore, the flex in some areas may be increased by adding a different yarn every eighth course in sections where no second yarn was present. The use of every 4th and 8th course in the plaiting structure is for illustrative purposes only. The plaiting structure can range from every other course to every 9th course using machines, such as, but not limited to, models SFG-I, NSFG, and SWG, manufactured by Shima Seiki Mfg., Ltd.

The perspiration and moisture wicking properties of the wound care foot wrap and outer dressing, such as the wound care foot wrap 102, the toe separator 402, and outer dressing, may also be achieved as disclosed in commonly assigned U.S. Provisional Ser. No. 61/571,569, and U.S. patent application Ser. No. 13/538,368, which are incorporated herein by reference in entirety. This technology includes a super absorbent material comprised of an electrospun polyurethane and bound acrylate. One such super absorbent material is marketed as SNS Nanosorb® 28. SNS Nanosorb® 28 has a higher affinity for water compared with the nylon 6,6 of the inner knitted layer. The super absorbent material pulls in moisture wicked to it by the nylon yarn, which the super absorbent material subsequently pulls into its internal matrix. Such moisture movement leaves the nylon of the inner knitted layer dry, keeping the moisture away from a user's skin. Different thicknesses of the super absorbent material may be employed. Moreover, the super absorbent material can be used, in lieu of additional padding, to protect body parts from inadvertent bangs and bumps. Moisture absorption is enhanced, in embodiments of the invention, by including poly-acrylates, polyurethanes, polyvinyl alcohol, hydrogels, and other hydrophilic materials.

Embodiments of the invention, discussed herein, are directed towards foot wraps. It is to be further understood that other articles for wound care on other parts of the body are contemplated herein, such as hands, knees, elbows, ankles, and the like, which may contain all features of embodiments of the invention. Moreover, a wound care foot wrap may be combined with a substantially cylindrical compression sleeve for the neck of a patient, as could be manufactured by knitting the two components together in a single knitting operation, to form an article that addresses more than one body part.

Outer dressings, which are placed over articles, such as foot wraps in accordance with embodiments of the invention, are further contemplated herein. Outer dressings are placed over the inner article, the skin-contacting article. The inner article can remain on the wound, for example a burn for an extended period of time, e.g., weeks, while the outer dressing might be changed every day. A burn patient can self-dress a wound easily and effectively once they were sent home from a hospital. As discussed below, this approach could be applied to gloves, finger cots, face masks and others articles.

Embodiments according to the invention further comprise methods for treating wounds. For example, at least one exemplary method for treating a wound, comprises wrapping a knitted fabric layer having a plurality of flaps comprising at least one metalized covered yarn and fasteners disposed on the plurality of flaps, wherein the knitted fabric layer is adapted to form a heel pocket and wrap around at least one of a foot or a lower leg area of a patient to form a foot wrap and the fasteners on the plurality of flaps engage the knitted fabric layer so that the foot wrap can be releasably tightened and loosened and wherein the metalized yarn has anti-microbial properties and promotes the wicking of moisture away from a wound.

The method of treating a wound further comprises a plurality of flaps that are optionally adjustably tight around a toe area, looser in an ankle area, and even looser in a lower leg area, thereby creating a graduated compression from the toe area to the lower leg area. The method further comprises wrapping an outer dressing around the wound care foot wrap.

All ranges of numerical values for any dimension recited herein are exemplary, are not to be considered limiting, and include ranges therebetween, and can be inclusive or exclusive of the endpoints. Optional included ranges can be from integer values therebetween, at the order of magnitude recited or the next smaller order of magnitude. For example, if the lower range value is 0.1, optional included endpoints can be 0.2, 0.3, 0.4 . . . 1.1, 1.2, and the like, as well as 1, 2, 3 and the like; if the higher range is 8, optional included endpoints can be 7, 6, and the like, as well as 7.9, 7.8, and the like.

While the foregoing is directed to embodiments of the invention, other embodiments of the invention may be devised without departing from the scope thereof, and the scope thereof is determined by the following claims.

1. A wound care foot wrap, comprising:
a knitted fabric layer comprising a wicking yarn and a metalized yarn and having a main area and a heel portion, wherein a toe flap, a foot flap, an instep flap, and a lower leg flap extend from the main area;
at least one fastener disposed on each of the foot flap, the toe flap, and the instep flap;
wherein the knitted fabric layer is adapted to form a heel pocket and wrap at least one of a foot or a lower leg area of a patient to form a wrap and the fasteners on the foot flap, the toe flap, and the instep flap engage the knitted fabric layer so that the foot wrap can be releasably tightened and loosened.

2. The wound care foot wrap of claim 1, wherein the knitted fabric layer comprises at least one covered yarn having a metalized yarn having at least one of copper, silver, gold, or copper coated thereon.

3. The wound care foot wrap of claim 1, wherein the metalized yarn comprises a hydrophilic yarn.

4. The wound care foot wrap of claim 1, wherein the metalized yarn comprises nylon, polyester, rayon, or any combination or blend thereof.

5. The wound care foot wrap of claim 4, wherein the nylon yarn comprises at least one of nylon 6, nylon 6,6 or a wicking nylon yarn having an irregularly shaped cross section.

6. The wound care foot wrap of claim 2, wherein the at least one covered yarn comprises an elastomeric yarn core, a first cover of cotton, and a second cover of a metalized nylon yarn.

7. The wound care foot wrap of claim 6, further comprising a metalized nylon yarn plaited with the at least one covered yarn to form a wound care foot wrap comprising a second layer.

8. The wound care foot wrap of claim 7, further comprising an antimicrobial agent disposed on or in at least one of the knitted fabric layer or a plaited yarn.

9. The wound care foot wrap of claim 8, wherein the antimicrobial agent is at least one of triclosan, 2-propanol, n-halamines, polymeric biguanides, quaternary ammonium compounds, chlorhexidine gluconate, silver-zinc and silver-copper zeolites, or compounds and combinations thereof.

10. The wound care foot wrap of claim 1, further comprising a moisture reservoir fluidly coupled with the knitted fabric layer.

11. The wound care foot wrap of claim 10, wherein the moisture reservoir further comprises an electrospun polyurethane and bound acrylate.

12. The wound care foot wrap of claim 10, wherein the moisture reservoir comprises at least one of polyacrylates, polyvinyl alcohol, or hydrogel, hydrocolloids, or hydrophilic particles.

13. The wound care foot wrap of claim 1, further comprising an outer dressing to cover the knitted fabric layer.

14. The wound care foot wrap of claim 13, wherein the outer dressing comprises a sock knitted from at least one covered yarn further comprising an elastomeric yarn core, a first cover of cotton, and a second cover of a metalized nylon yarn.

15. A kit, comprising:
the wound care foot wrap of claim 1;
a toe separator; and
an outer dressing for covering the wound care foot wrap.

16. The kit of claim 15, wherein the outer dressing comprises at least one of cotton yarns, nylon yarns, elastomeric yarns, rayon yarns, or a blend or a mixture thereof.

17. The kit of claim 15, wherein the toe separator comprises at least one covered yarn having an elastomeric yarn core, a first cover of cotton, and a second cover of a metalized nylon yarn.

18. A method for treating a wound, comprising:
wrapping a knitted fabric layer having a plurality of flaps comprising at least one metalized covered yarn and fasteners disposed on the plurality of flaps,
wherein the knitted fabric layer is adapted to form a heel pocket and wrap around at least one of a foot or a lower leg area of a patient to form a foot wrap and the fasteners on the plurality of flaps engage the knitted fabric layer so that the foot wrap can be releasably tightened and loosened and wherein the metalized yarn has anti-microbial properties and promotes the wicking of moisture away from a wound.

19. The method of claim 18, wherein the plurality of flaps are adjustably tight around a toe area, looser in an ankle area, and even looser in a lower leg area, thereby creating a graduated compression from the toe area to the lower leg area.

20. The method of claim 18, further comprising the step of wrapping an outer dressing around the wound care foot wrap.
